(19) United States
(12) Patent Application Publication
(10) Pub. No.: US 2004/0186371 A1
(43) Pub. Date: Sep. 23, 2004

(54) Medical image processing apparatus and medical network system

(75) Inventor: Haruyuki Toda, Tokyo (JP)

Correspondence Address: FRISHAUF, HOLTZ, GOODMAN & CHICK, PC, 767 THIRD AVENUE, 25TH FLOOR, NEW YORK, NY, 10017-2023, US

(73) Assignee: KONICA MINOLTA HOLDINGS, INC., Tokyo (JP)

(21) Appl. No.: 10/800,316

(22) Filed: Mar. 11, 2004

(30) Foreign Application Priority Data
Mar. 20, 2003 (JP) JP2003-078079

(51) Int. Cl. A61B 5/05

(52) U.S. Cl. 600/407

(57) ABSTRACT

A medical image processing apparatus for receiving medical image information from another apparatus, performing an image processing and sending a medical image to another device to display, including: a first specifying tool for specifying a first display order of the medical image; a second specifying toll for specifying information to be used for determining a display order of the medical image on a display terminal; and a correcting tool for correcting the information to be used for determining the display order to conform to the first display order.

### BACKGROUND OF THE INVENTION

The present invention relates to a medical image processing apparatus for receiving medical image information, processing the image and sending it to another apparatus; and a medical network.

A radiation image is often used for medical diagnosis. A commonly known apparatus to get this radiation image is a medical image generating apparatus wherein, after a plate-like stimulable phosphor has absorbed the radiation, coming from a radiographing modality radiation emitting section of CR (computed radiography) and having passed through the subject, the stimulable phosphor is excited by scanning of laser beam. Thus, the radiation energy (radiation image information) stored by this stimulable phosphor through absorption emits light as fluorescent light, which is subjected to photoelectric conversion to get a radiation image signal, whereby radiation image is read out in this apparatus. The fluorescent light emitted from the stimulable phosphor plate is collected by scanning of laser beam and is subjected to photoelectric conversion by a photomultiplier. This electric signal is amplified, and image processing is applied in conformity to the digital signal gained by conversion from this analog signal, whereby radiation image is read out to generate medical image information. (Refer the following Patent Document 1, for example)

According to the prior art, the medical image information read and generated by a medical image generating apparatus as described above is sent to an image processing apparatus and is outputted to an external output device such as

an image display device (viewer) or an image storage device (server) after having been subjected to predetermined image processing. In this case, information for identifying the order of images is determined for each image display device (viewer) or image storage device (server), and images are displayed arranged in that order. (See the following Patent Document 2, for example)

In the viewer and server, however, the order of images to be displayed is determined according to the information attached to the received image, and initial display is given. Thus, if the attached information at the time of reception fails to conform to the order of display, initial display is different from what is preferred by an operator.

Further, the order suitable to the generation of images by an image generation apparatus does not always match that suitable to the observation of images on the image display device. If the information attached at the time of reception is used directly, the order of display will not be satisfactory. For example, when an orthopedist diagnoses images in the examination of the lumbar spine in four directions, he wants to view images in the order of front face, side face, right oblique and left oblique. However, radiographing is performed in the order of front face, right oblique, side face and left oblique suitable to the shift of the patent's body. Thus, when the image display device has determined the order of display according to the time of radiographing or image signal numbers, images are not arranged in the order desired by the orthopedist.

In cases described above, images must be rearranged subsequent to initial display or diagnosis must be started when images are not arranged in the optimum order. In the former case, unwanted operation is required to cause loss of time and effort. In the latter case, diagnosis must be started when images are not arranged in the order desired by the orthopedist.

[Patent Document 1]

Japanese Application Patent Laid-Open Publication No. Hei 11-344783

[Patent Document 2]

Official Gazette of Japanese Patent 3028980

In view of the problems involved in the prior art described above, it is an object of the present invention to provide a medical image processing apparatus, a medical network system and a program for medical image processing apparatus.

### SUMMARY OF THE INVENTION

To achieve the aforementioned object, the first medical image processing apparatus of the present invention receives medical image information from another device (information source), processes the image and sends it to another device (display terminal), and comprises:

a first specifying tool for specifying a first display order of the medical image;

a second specifying tool for specifying information to be used for determining a display order of the medical image on a display terminal; and

a correcting tool for correcting the information to be used for determining the display order to conform to the first display order.

The aforementioned medical image processing apparatus specifies the order of the images, and corrects the information used to determine the order of display at the display terminal in such a way that the order of display will conform to that specified. Thus, it is possible to remove the difference between the order suitable for generation of images by an image generation apparatus at the source of reception and that suitable to the observation of images on the image display device at the display terminal, and to display images in an proper order.

To achieve the aforementioned object, the second medical image processing apparatus of the present invention receives medical image information from another device (information source), processes the image and sends it to another device (display terminal), and comprises:

means (a display) for displaying the received image in the form of a thumbnail or list for each examination;

means (a specifying tool) for sorting the aforementioned displayed thumbnail or list and specifying the order;

means (a specifying tool) for specifying information used to determine the order of display at a display terminal; and

means (a correcting tool) for correcting the aforementioned information used to determine the order of display so that it will conform to the order specified by sorting of the thumbnail or list.

The aforementioned medical image processing apparatus sorts the displayed thumbnail or list, specifies the order of the images, and corrects the information used to determine the order of display at the display terminal in such a way that the order of display will conform to that specified by sorting. Thus, it is possible to remove the difference between the order suitable for generation of images by an image generation apparatus at the source of reception and that suitable to the observation of images on the image display device at the display terminal, and to display images in an proper order, without changing the order of generation by the image generating apparatus (e.g. radiographing order) or the order of display by the image display device. This feature ensures quick diagnosis and proper diagnosis in a manner that facilitates doctor's diagnosis.

The third medical image processing apparatus of the present invention receives medical image information from another device (information source), processes the image and sends it to another device (display terminal), and comprises:

means (a specifying tool) for specifying the permission of starting image output for each image;

means (a specifying tool) for specifying information used to determine the order of display at the display terminal;

means (a correcting tool) for correcting the information used to determine the order of display so that images are arranged in the order in which the permission of starting image output is given in the same examination, when the permission of starting image output has been specified.

The aforementioned medical image processing apparatus specifies the information used to determine the order of display at the display terminal, and corrects the information used to determine the order of display so that the images are arranged in the order in which the permission of starting image output is given in the same examination, when the permission of starting image output to the display terminal has been specified. This makes it possible to remove the difference between the order suitable for generation of images by an image generation apparatus at the source of reception and that suitable to the observation of images on the image display device at the display terminal, and to display images in an proper order, without changing the order of generation by the image generating apparatus (e.g. radiographing order) or the order of display by the image display device. This feature ensures quick diagnosis and proper diagnosis in a manner that facilitates doctor's diagnosis.

A fourth medical image processing apparatus of the present invention receives medical image information from another device (information source), processes the image and sends it to another device (display terminal), and comprises:

means (a tool) for receiving request information and determining the order of the image requested in the examination, from the aforementioned request information received;

means (a tool) for specifying the information used to determine the order of display at the display terminal; and

means (a tool) for correcting the information used to determine the order of display so that images are arranged in the order specified by the aforementioned request information.

This medical image processing apparatus determines the order of the image requested from the aforementioned request information received, specifies the information used to determine the order of display at the display terminal; and corrects the information used to determine the order of display so that images are arranged in the order specified by the aforementioned request information. This makes it possible to remove the difference between the order suitable for generation of images by an image generation apparatus at the source of reception and that suitable to the observation of images on the image display device at the display terminal, and to display images in an proper order, without changing the order of generation by the image generating apparatus (e.g. radiographing order) or the order of display by the image display device. This feature ensures quick diagnosis and proper diagnosis in a manner that facilitates doctor's diagnosis.

The request information can be acquired from the request information management apparatus such as RIS or HIS, using the modality work list (DICOM code). It is also possible to make such arrangements that request information is acquired from the request information management apparatus such as RIS and HIS using the modality work list (DICOM code), and the request corresponding to the image at the time of selecting images is determined.

The fifth medical image processing apparatus of the present invention receives medical image information from another device (information source), processes the image and sends it to another device (display terminal), and comprises:

means (a tool) for storing a condition file where information for image processing and outputting of an image file is set in advance;

means (a tool) for presetting the order of images in the aforementioned condition file;

means (a tool) for determining a condition file to be used, for a received image, from among a group of the aforementioned condition files;

means (a tool) for specifying the information used to determine the order of display at the display terminal; and

means (a tool) for correcting the information used to determine the order of display so that images are arranged in the order specified by the condition file.

This medical image processing apparatus presets the order of images in the condition file, specifies the information used to determine the order of display at the display terminal, and corrects the information used to determine the order of display so that images are arranged in the order specified by the condition file. This makes it possible to remove the difference between the order suitable for generation of images by an image generation apparatus at the source of reception and that suitable to the observation of images on the image display device at the display terminal, and to display images in an proper order, without changing the order of generation by the image generating apparatus (e.g. radiographing order) or the order of display by the image display device. This feature ensures quick diagnosis and proper diagnosis in a manner that facilitates doctor's diagnosis.

In the first through fifth medical image processing apparatuses, the information on series is then changed after the order of display at the display terminal has been sorted according to the information on the site to be diagnosed. This method allows correction of the information used to determine the order of display so that images are arranged in the order specified by a series number when the order of display at the display terminal is determined according to series number.

The medical network system comprising: any one of the aforementioned first through fifth medical image processing apparatuses; a medical image generating apparatus for generating medical image information and sending to the medical image processing apparatus; and a display device for displaying the image information having undergone image processing, from the medical image processing apparatus.

This medical network system is capable of removing the difference between the order suitable for generation of images by an image generation apparatus and that suitable to the observation of images on the image reserving device or image display device and to display images in an proper order, without changing the order of generation by the image generating apparatus (e.g. radiographing order) or the order of display by the image display device. This feature ensures quick diagnosis and proper diagnosis in a manner that facilitates doctor's diagnosis.

### BRIEF DESCRIPTION OF THE DRAWINGS

FIG. 1 is a block diagram of the configuration of a medical network system as an embodiment of the present invention;

FIG. 2 is a block diagram of the configuration of a medical image processing apparatus 2 in FIG. 1;

FIG. 3 is a flowchart representing the first operation in the medical image processing apparatus as an embodiment of the present invention;

FIG. 4 is a flowchart representing the second operation in the medical image processing apparatus as an embodiment of the present invention;

FIG. 5 is a flowchart representing the third operation in the medical image processing apparatus as an embodiment of the present invention;

FIG. 6 is a flowchart representing the fourth operation in the medical image processing apparatus as an embodiment of the present invention;

FIG. 7(a) is a diagram showing an example of the order of radiographing in an embodiment of the present invention; and

FIG. 7(b) is a diagram showing an example of the order of sorting the images radiographed in the order given in FIG. 7(a).

### DETAILED DESCRIPTION OF THE PREFERRED EMBODIMENT

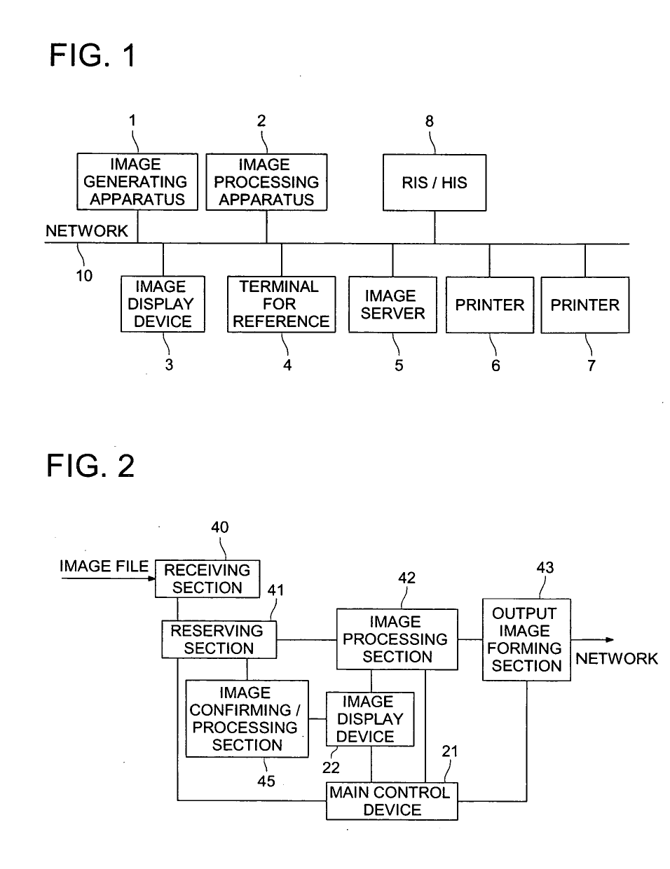

The following describes the medical network system as an embodiment of the present invention with reference to drawings: FIG. 1 is a block diagram of the configuration of a medical network system as an embodiment of the present invention.

The medical network system of FIG. 1 comprises:

a medical image generating apparatus 1 for causing light to be emitted by scanning, with excitation light, the stimulable phosphor panel recording the information on radiation image of a subject (patient), and for generate the medical images by the radiographing modality of the CR (computed radiography) for getting image-information through photoelectric conversion of the light;

a medical image processing apparatus 2 for applying image processing and outputting the image information after an image file has been input through the medical image generating apparatus 1; and

an image display device (viewer) 3, consisting of a personal computer or a workstation, used by a radiologist to perform diagnosis by referring to the display.

The medical network system of FIG. 1 comprises:

a terminal for reference 4, consists of a personal computer and workstation, used for making reference to images, without diagnosis being performed, inferior in image quality including resolution than the image display device 3;

a image server 5, consisting of a personal computer and workstation, for storing the image file in an image database, and for searching and reading out images from the image display device 3 and terminal for reference 4; and

a plurality of printers 6 and 7 for outputting the image data from the image generating apparatus 1 or image processing apparatus 2, into a recording medium including a film or paper as visible images. The medical network system shown in FIG. 1 is connected with a RIS/HIS (request information management apparatus) 8 for request management.

The medical network system shown in FIG. 1 is on-line connected with devices 1 through 8 through a network 10, and is designed to exchange information with each other.

Next, the following items A-H relating to medical image generating apparatus 2 in FIG. 1 will be explained in detail successively.

A. Apparatus structure

B. Information

C. File

D. Input and display of main information

E. Image confirming procedures

F. Output

G. Output image formation

H. Utility function

A. Apparatus Structure

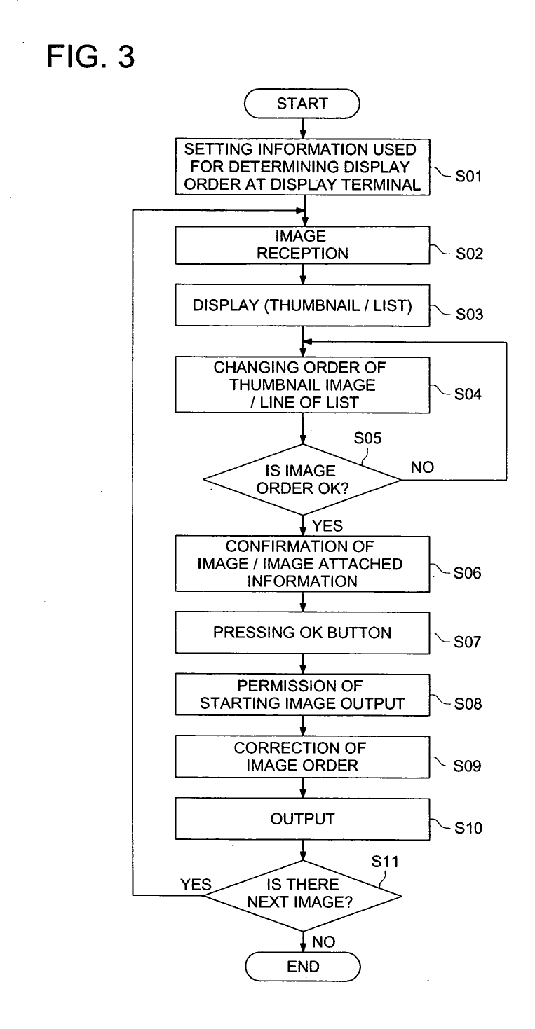

FIG. 3 is a block diagram showing the structure of medical image processing apparatus 2.

a. The medical image processing apparatus 2 shown in FIG. 2 is provided with main control apparatus 21 that controls operations of the whole radiographing system for radiographic images, and with image display device 22 that is composed of a CRT display or of a liquid crystal panel and displays digital image data obtained by medical image generating apparatus 1, and it can be composed of a personal computer and includes information inputting device such as an input key board and a mouse.

As shown in FIG. 2, the image processing apparatus 2 further includes receiving section 40 that receives image files from image forming apparatuses 1 and 1a, preserving section 41 that is composed of a hard disc or RAM and stores various pieces of information such as an order of received image files and output queue, image processing section 42 that conducts image processing for image information in the image files, output image forming section 43 that forms output images to be outputted to the outer apparatuses, and image confirming/processing section 45 that makes image display device 22 to display reduced images for confirmation of received images. Main control apparatus 21 controls each of sections 40-43, 45 and the display device 22.

b. The functions of the medical image processing apparatus 2 are as follows, and each function is controlled by the main control apparatus 21.

1) To receive image files from medical image generating apparatus 1 or the like at receiving section 40.

2) To preserve image files in preserving section 41 temporarily.

3) To confirm image quality by the use of reduced images prepared by image confirming/processing section 45.

4) To conduct image processing on the image processing section 42.

5) To form output images at output image forming section 43.

6) To transmit output images to outer apparatuses such as image server 5 and printers 6-8 through the network 10.

B. Information

Information handled by the medical image processing apparatus 2 can be classified into the following five types of information.

a. Condition Information

The condition information is one necessary for receiving image files, and for outputting to an outer apparatus such as image server 5 as a processed image file, and it includes the following.

(a) Image Processing Information

This is information relating to gradation processing and frequency processing in image processing section 42.

(b) Outputting Apparatus Information

This is information relating to outer outputting apparatuses such as image server 5 which reproduces and outputs image data, and it designates an area to be outputted, magnification and rate of reduction, output format (multi-format, split radiographing format), overlay and existence of gradation processing and frequency processing for each outputting apparatus such as image server 5.

(c) Overlay Information

This is information about existence and position of overlay such as AP/PA, R/L and comment.

(d) Information About Specific Designation

Information of protection: Image files are preserved until a protection is removed, even after the mage transmission.

Information of pending: Transmission is reserved. This is designated when transmission is needed after the image is reviewed.

Information of priority (emergency): This is designated when preferential output such as emergency radiographing is required. This is registered to be at the forefront of a cue.

b. Patient Information

This is information concerning patients.

(a) Patient ID Information

An ID number, a full name, the distinction of sex and the date of birth of a patient.

(b) Request Information

This is information with which a doctor requests radiographing, which includes information relating to patient conditions and instructions for the date and method for the inspection requested.

c. Information of Radiographing Implementation

This is information concerning the results of receiving and image processing.

(a) The Results of Receiving and the Date of Radiographing are Included.

(b) Results of Image Processing

This is a result of calculation of image processing parameter, and image data are processed based on this result when outputting.

(c) System Information

This includes a part of system information such as a system structure at the point of time when radiographing is conducted.

d. System Information

(a) Information for controlling the system in FIG. 1

(b) Structures of system in FIG. 1 (outer apparatus such as image server 5 connected, and its name)

(c) Parameter and table to control equipment constituting the system in FIG. 1

(d) Setting information concerning medical image generating apparatus 1 representing an inputting apparatus

(e) Setting information concerning an outputting apparatus such as information of imager 6 and HOST information

e. Image Data

(a) Image data received from medical image generating apparatus 1

(b) Reduction image data for display prepared from image data for image confirmation.

(c) Reduction image data for image processing for image processing of reduction image for display at image confirming/processing section 45.

(d) Output image data which have been subjected to gradation processing and frequency processing.

C. File

A file handled by medical image processing apparatus 2 is preserved in preserving section 41, and it is classified into the following seven files.

a. Condition File

A condition key is a key for setting in advance image processing conditions for image files and outing conditions. It has a condition file that corresponds to each condition key. The condition file is composed of the radiographing information above mentioned. It is classified in terms of radiographing regions (lung, abdomen, head and others), radiographing posture (standing posture, lying posture and others), radiographing direction (front, side and others), characteristics of a patient (the distinction of sex, age, physical structure and others), the name of a disease and a radiologist, and a name and radiographing information corresponding to each of them are established in advance. Main control apparatus 21 establishes a condition file group for each of classified plural classifications, then, sets plural condition files for each condition file group thus established, and preserves in preserving section 41. The optimum condition is selected in the course of receiving images.

b. Image Header File

After receiving, an image header file is prepared. The header file is composed of a reservation file of the radiographing (namely, radiographing information, patient information) and information of conducting radiographing. When a user refers to radiographing information, patient information and information of conducting radiographing for changing, the user refers to an image header file.

c. Reduction Image File

This represents image data which are obtained by reducing image data at a certain reduction ratio.

(a) Reduction Image Data for Display

This reduction image for display is used by data displayed on image display device 22 in FIG. 3.

(b) Reduction Image Data for Image Processing

This represents reduction image data for calculating a parameter that is for conducting image processing. The reduction rate is determined so that a length of one pixel after reduction may be the same as a length designated in advance. Due to this, it is possible to correct the difference in a size of a pixel to be read with the image after being reduced. Calculation of a parameter for image processing is conducted by the reduction image for image processing, and image data are not used.

d. Image File

(a) An image file is composed of image-accompanying information (image header) and image data.

(b) An image header is composed of condition information, patient information and implementation information. When a user makes a change by referring to condition information, patient information and implementation information, the user refers to the image header.

e. Output Image File

This is a file of output image data which have been subjected to the designated processing among frequency processing, gradation processing, overlay, rotation and enlargement and reduction.

f. System File

This is one wherein the system information mentioned above is made to be of a type of a file.

D. Input and Display of Primary Information

a. Received Image Information Display

Received images are displayed on a thumbnail mode.

b. Output Information Display

1) An outputting size, a direction, a trimming position, an outputting position and a method of enlargement and reduction are designated, and registered in a condition file in advance.

2) When a condition key is selected, the output area and output image area are determined under the condition designated in advance, and are displayed on the screen of the image display device 22. A size of the output area display area on the screen of the image display device 22 is made to be the maximum output area in outputting. An output area and an output image area are displayed graphically on the output area display area. Due to this, the appropriate output area and the output image area can be selected and confirmed.

c. Overlay Information

1) It is designated whether or not “AP”, “PA”, “R”, “L”, comment and a division are overlaid, or where they are overlaid, and they are registered in a condition file in advance.

2) Output images are displayed on an output area display area on the screen of the image display device 22, and overlay information is displayed graphically there.

3) It is possible to select an appropriate overlay and to designate a position.

4) It is possible to confirm that a portion screened by the overlay to be invisible is not present. When the overlay causes troubles on diagnoses, it can be moved.

d. Inputting and Outputting of On-Line Information from RIS

1) Orders from a doctor are inputted. The orders thus inputted are converted into the format of this system to be preserved in the reservation file. The radiographing region and radiographing method are converted into corresponding radiographing conditions.

2) An image header file is converted into the format on the RIS side, and outputted.

e. Image List

An image file can be displayed as a list.

E. Image Confirming Procedures

a. Operations of System in Image Confirmation

(1) An image file is received from medical image generating apparatus 1, and is stored in preserving section 41.

(2) The image file stored in a storage medium of the preserving section 41 is reduced at the reduction rate designated by image confirming/processing section 45 in advance.

(3) Reduction images are displayed successively on the screen of image display device 22.

(4) After receiving and completion of display, digital image information is subjected to image processing in the method designated in advance by a radiographing condition key, and is displayed again on image display device 22. Reduction images are used to determine a parameter for image processing.

(5) Images which are displayed successively on the image display device 22 and are subjected to gradation processing after being displayed are displayed again.

(6) When an operator observes received images displayed on the image display device 22 and judges that they are normal images, a key for confirming the completion of receiving is inputted from a character information inputting apparatus, thus, image confirmation is completed.

(7) When patient information, image processing methods and output methods need to be changed, it is possible to input new information from a character information inputting apparatus.

(8) When an image confirming key is pressed, image confirmation for that image is completed, and the following image is displayed automatically.

(9) When an image has a problem, it is possible to change image processing. By reserving it, it is possible to change image processing in detail later.

(10) When an image confirming key is inputted, image confirmation is completed, and the following processing is conducted.

1) The image file is preserved in preserving section 41 as a confirmed image file.

2) Images whose image confirmation has been finished are registered in a cue for outputting to an outer apparatus.

3) Then, the received image file is displayed so that images may be confirmed.

(11) When a reservation key is inputted, image confirmation is completed.

F. Output

1) Output is conducted on a non-synchronization basis with image confirmation.

2) Though a cue is made and controlled for each outer apparatus, plural printers 6-8, for example, are sometimes controlled by one as will be stated later.

3) The whereabouts of the image in terms of registration in a cue of an outer apparatus is preserved in preserving section 41 as a cue registration table, and it is updated and controlled for each registration and cancellation.

4) An image registered in the cue is outputted to an outer apparatus in the order of registration, and the image whose outputting is finished is deleted from the cue.

5) When carrying out outputting, an image file stored in preserving section 41 is specified from numbers registered in the cue.

6) An output image is formed under the condition preserved in the image file. The image header is converted into the format determined for each outputting apparatus, and is transmitted together with image data.

G Forming Output Image

a. An output image is formed by output image forming section 43 mainly through the following processing.

1) Image data are read from preserving section 41 to a memory for images.

2) Frequency processing is conducted.

3) Equalization processing is conducted.

4) Gradation processing is conducted.

5) Rotation of an image is carried out.

6) Mirror reversing is conducted.

7) Enlargement and reduction are carried out.

8) overlay is carried out.

b. With respect to each of 2)-8), whether it is executed or not can be designated by condition information for each outputting apparatus.

c. It is possible to designate that image data subjected to designated processing of each of 2)-8) are preserved as processed image data file. Re-processing of common processing section of output image for each output apparatus is removed.

d. For example, when an enlargement rate and a reduction rate of an output image for each output apparatus are different from others, if images which have been subjected to processing up to 6) are preserved, it is possible to shorten a period of time for 2)-6) by reading images which have been subjected to processing up to 6) and by processing and transferring only 7) and 8), when transmitting to another apparatus.

e. Processing 5) and 6) are conducted simultaneously with either one of 2), 3) and 4). Access of memory is reduced, and processing time can be shortened.

H. Utility Function

a. As a utility for a user, some functions are provided thereto. Utility function is restricted by a password for each of general user, a manager and a maker. In particular, for a change of information relating to images, a password of a manager is required for security.

b. Image File Operation

1) An image file list is displayed, and information concerning images preserved is displayed on image display device 22 in the order of receiving.

2) When a desired image is selected from the list, patient information, condition information and images are displayed in the same form as in the screen in the case of image confirmation.

3) Patient information, image processing methods and outputting methods can be changed.

4) With regard to the image designated to be “reservation” in the case of radiographing, the “reservation” can be canceled by reconfirming here.

5) The order of outputting can be changed, including whether outputting to each outer apparatus is conducted or not.

c. Radiographing Record, Emission Record

1) Radiographing information and patient information are processed statistically, and are provided to a user as a radiographing record and an emission record.

2) The number of shots per each radiographing region for a designated period and a list of radiographing conditions for shots per day can be outputted.

d. Customizing

A screen and operating procedures can be customized for each user.

The following describes the first through fourth operations (1) through (4) for outputting the images after changing the order of images in the process of one examination in such a way that images can be sorted, with consideration given to the order of display on the image display device 3 for diagnosis in the image processing apparatus 2 shown in FIGS. 1 and 2.

(1) Referring to the flowchart of FIG. 3, the following describes the case where images are sorted on the thumbnail or list display:

The first step is to set the information for determining the order of display on the display terminal (image display device 3 of FIG. 1) (S01). To put it another way, it is the step of using what type of information should be used to determine the order of display. For example, the common information item includes an image number, series number and the date of radiographing. Thus, any one of the image number, series number and the date of radiographing is selected from the combination box.

The next step is to receive then image information from the image generating apparatus 1 of the source of reception in FIG. 1 (S02). The received image is given in thumbnail or list display for each examination (S03). The thumbnail image or list line is dragged by a pointing device, whereby the order is changed (S04).

If the order of image display on the thumbnail or list display has agreed with the desired order (S05), the operator confirms the image and image attached information (patient/examination information) (S06). If the [OK] button is pressed on the screen (S07), permission of starting image output is specified for each examination (S08).

For example, when an image number is set as information used to determine the order of display, image numbers 1, 2 and 3 are corrected in the ascending order (or descending order) of the thumbnail or list display (1, 2, 3, . . . in that order) (S09), thereby outputting images (S10). If there is next image information (S11), the system goes to step S02 and the same procedure is repeated.

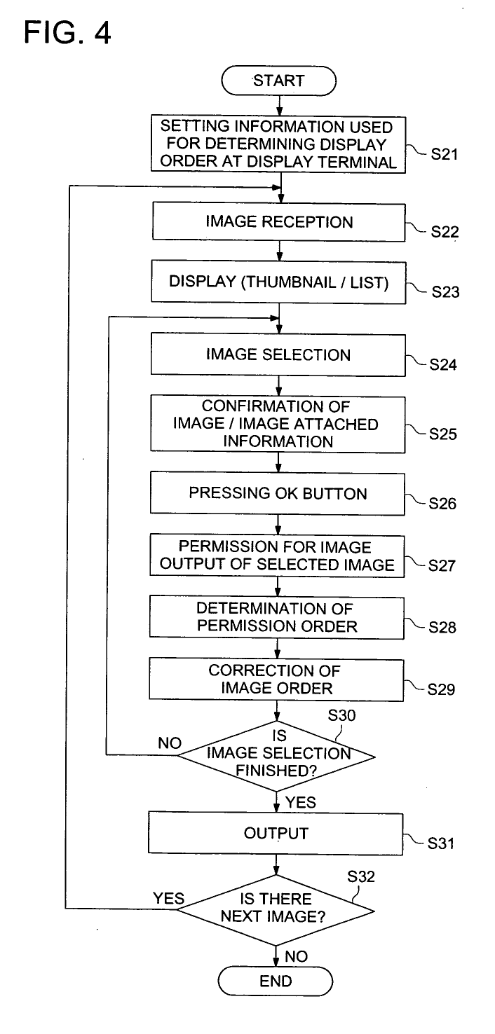

(2) Referring to the flowchart of FIG. 4, the following describes the case of sorting in terms of image:

The first step is to set the information for determining the order of display on the display terminal (image display device 3 of FIG. 1) (S21). Then the image information is received from the image generating apparatus 1 as a source of reception in FIG. 1 (S22) and the received image is given in thumbnail or list display for each examination (S23). The thumbnail image or list line is clicked by a pointing device, whereby the image is selected (S24).

The operator verifies the image and image attached information (patent/examination information) (S25). If the [OK] button is pressed on the screen (S26), permission of starting image output is specified for the selected image (S27)

When permission of starting image output has been issued, a decision is made to determine the ordinal position of examination item, containing the image, where the permission of starting image output has been specified (S28). For example, if the third item contains the image for which the permission of starting image output is specified, and an image number is set as information used to determine the order of display, the image number is corrected as “3” (S29).

When the image is further selected (S30), the system goes back to step S24, and the same procedure is repeated. Upon termination of the image (S30), each image is outputted (S31). If there is the next image information (S32), the system goes back to step S22, and the same procedure is repeated.

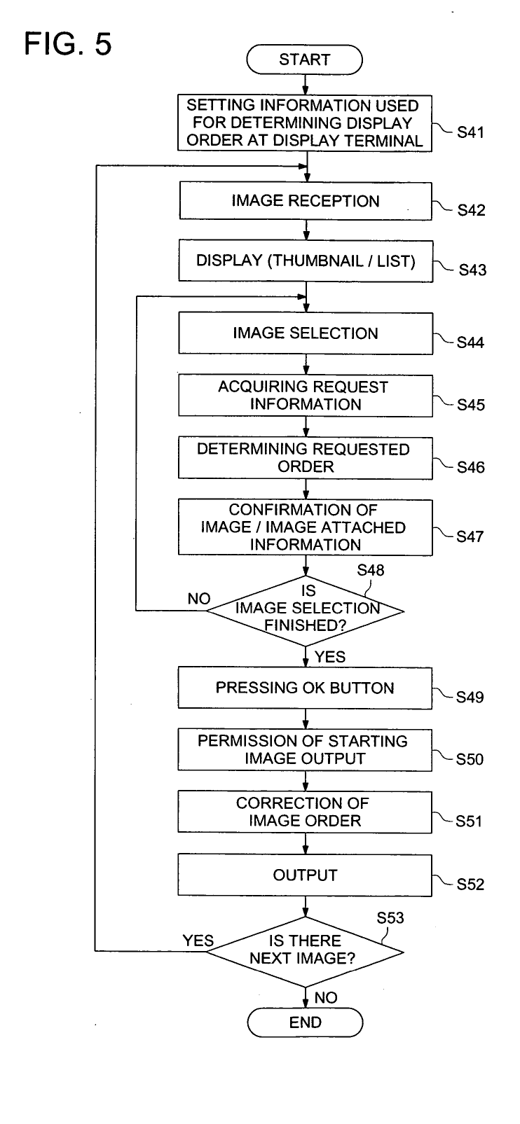

(3) Referring to the flowchart of FIG. 5, the following describes the case where the order of request is sorted in comparison with the request information:

The first step is to set the information for determining the order of display on the display terminal (image display device 3 of FIG. 1) (S41). Then the image information is received from the image generating apparatus 1 as a source of reception in FIG. 1 (S42) and the received image is given in thumbnail or list display for each examination (S43). The thumbnail image or list line is clicked by a pointing device, whereby the image is selected. (S44).

When the image has been selected, request information is obtained from the RIS/HIS (request information management apparatus) 8 using the modality work list (DICOM code), using the examination ID of the image or reception number as the key (S45).

Evaluation is made to determine the ordinal position of the request where the selected image has been radiographed among the requested items specified for each examination (S46). For example, when the request is made in the order of front face of lumbar spine, side face, right oblique and left oblique, the image where the site to be examined is located on the right oblique is determined to be the third.

It is also possible to make such arrangements that the request information is obtained from the RIS/HIS 8 in advance, using the modality work list (DICOM code), and the request corresponding to the image is determined at the time of selecting the image.

When the operator confirms the image and image attached information (patient/examination information) (S47), and selects the image further (S48), the system goes back to the Step S44, and the same procedure is repeated. If image selection has terminated for each examination (S48) and the OK button has been pressed on the screen (S49), the permission of starting image output is specified for the selected image (S50).

When the permission of starting image output, the image number is corrected (S51) if an image number has been set as information used to determine the order of display, and the image is outputted for each examination (S52). When there is next image information (S53), the system goes back to the Step S42, and the same procedure is repeated.

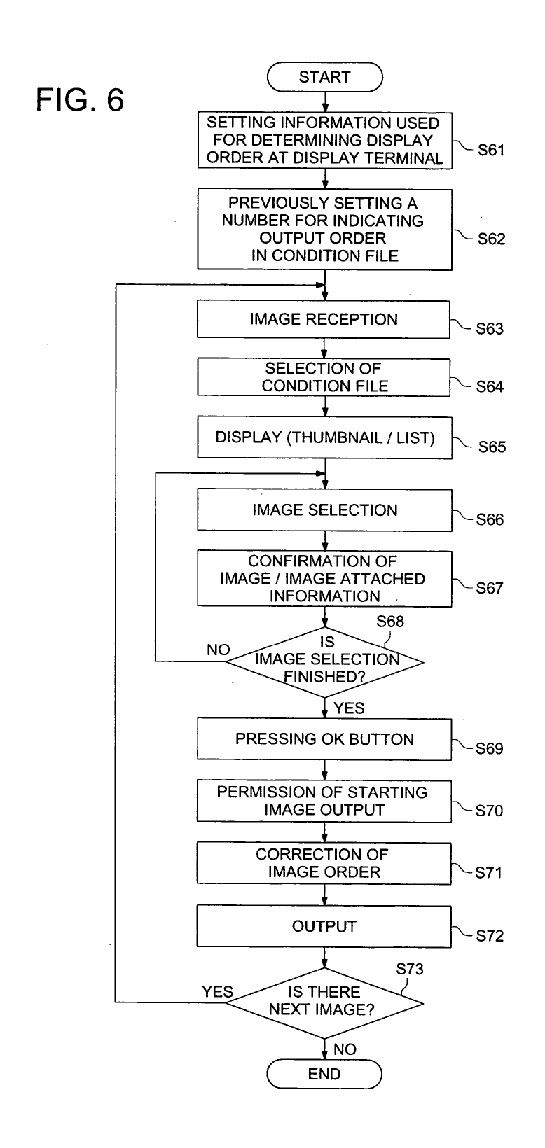

(4) Referring to FIG. 6, the following describes the case where sorting is performed by presetting to the condition file:

The first step is to set the information for determining the order of display on the display terminal (image display device 3 of FIG. 1) (S61). Then the number for specifying the order of output is set to the condition file in advance (S62). For example, “first display” is preset to the lumbar spine front face condition file, “second display” to the lumbar spine side face condition file, “third display” to the lumbar spine right oblique condition file, and “fourth display” to the lumbar spine left oblique condition file.

When an image has been received from the image generating apparatus 1 as a source of reception (S63), the condition file corresponding to that image is selected (S64), and the output format is determined. For example, in the case of an image of “lumber spine right oblique”, the lumber spine right oblique condition file is selected and the “Third display” is determined.

Then the received image is given in the thumbnail or list display for each display (S65). The image is elected by clicking the thumbnail image or list line with a pointing device (S66).

The operator confirms the image and image attached information (patent/examination information) (S67). When the image is further selected (S68), the system goes back to step S66, and the same procedure is repeated. If image selection has terminated for each examination (S68) and the OK button has been pressed on the screen (S69), the permission of starting image output is specified for the selected image (S70).

When the image number has been set as information used to determine the order of display according to the number for specifying the order of display preset for each condition file, the image number is corrected to “3” (S71) and the image is outputted for each examination (S72). If there is next image information (S73), the system goes to step S63 and the same procedure is repeated.

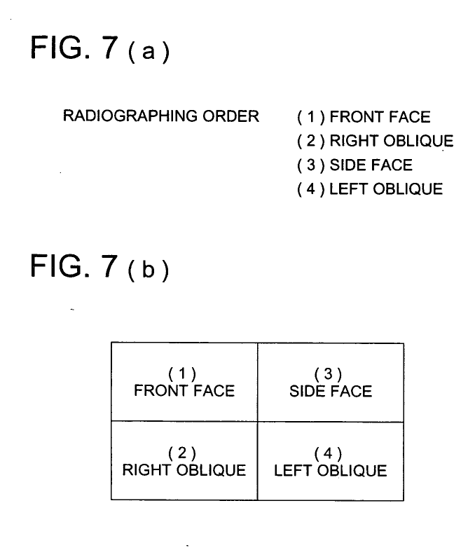

For example, when an orthopedist diagnoses images in the examination of the lumbar spine in four directions, radiographing is carried out in the order of (1) front face, (2) right oblique, (3) side face and (4) left oblique, as shown in FIG. 7(a). Accordingly, when the image display device 3 has determined the order of display in terms of radiographing time or image number, the images are displayed in the aforementioned order. By contrast, in FIGS. 3 through 6, images can be sorted in the order of (1) front face, (3) side face, (2) right oblique and (4) left oblique on the screen of the image display device 3, as shown in FIG. 7(b). Thus, images can be sorted in the order desired by the orthopedist.

As described above, the embodiment of the present invention removes the difference between the order suitable for generation of images by the image generation apparatus 1 and that suitable to the observation of images on the image display device 3. It also displays images in a proper order, without changing the order of generation by the image generating apparatus 1 (e.g. radiographing order) or the order of display by the image display device 3. This feature ensures quick diagnosis and proper diagnosis in a manner that facilitates doctor's diagnosis.

In FIGS. 3 through 6, it is also possible to make such arrangements that the permission of starting image output is specified by the operator using a mouse or keyboard. Alternatively, the permission of starting image output can be specified automatically after a lapse of predetermined period of time following reception of images.

Further, in FIGS. 4 through 6, it is also possible to make such arrangements that image selection is specified by the operator using a mouse or keyboard. Alternatively, it can be specified automatically in the order of image reception or according to preset information.

In FIGS. 3 through 6, outputting to other equipment such as an image display device can be performed immediately after the permission of starting image output; after images have been stored once; or in the order of registration in the queue. Actual outputting can be performed in the order subsequent to sorting according to the order of display, or the order prior to sorting.

In FIGS. 5 and 6, the order can be determined before the permission of starting image output is specified, or after it is specified. Further, in FIG. 5, association between the images and order thereof can be made at the time of image reception or image selection. Further, in FIG. 6, association between images and conditions files can be made at the time of image reception or image selection.

When the order of display in the image display device 3 is determined in terms of the series number, the order of display in the image display device 3 is classified according to the information on the site to be examined, and then the information on the series is changed. This allows correction of the information used to determine the order of display so that images are arranged in the order specified in terms of series number.

The following describes the case where the image display device 3 determines the order of display, for example, after the images have been classified according to the information on DICOM information series ((0020,000E) series interface UID (0020, 0011) series number, etc.) and information on the site to be examined ((0018, 0015), site to be examined, (0008, 2218) anatomical area sequence, etc.):

Let us assume that the examination site (0018, 0015) and series number (0020, 0011) of the images in the process of the same examination having been received are as shown in the following Table 1 or 2.

TABLE 1

| | (0018, 0015) | (0020, 0011) |
|---|---|---|
| 1st image | CHEST | 1 |
| 2nd image | ABDOMEN | 2 |
| 3rd image | CHEST | 3 |
| 4th image | BREAST | 4 |

TABLE 2

| | (0018, 0015) | (0020, 0011) |
|---|---|---|
| 1st image | CHEST | 1 |
| 2nd image | ABDOMEN | 1 |
| 3rd image | CHEST | 1 |
| 4th image | BREAST | 1 |

In the case of Table 1 or 2, images are classified according to the examination site (0018, 0015) and are then substituted into the series number (0020, 0011). This will give the order as shown in Table 3:

TABLE 3

| | (0018, 0015) | (0020, 0011) |
|---|---|---|
| 1st image | CHEST | 1 |
| 2nd image | ABDOMEN | 2 |
| 3rd image | CHEST | 1 |
| 4th image | BREAST | 3 |

The operations of the medical image processing apparatus 2 in the aforementioned embodiment of the present invention are controlled by a main control device 21 of FIG. 2 according to the program loaded in the memory section. Interface screen display for required information processing, inputting, determination, selection, etc. is carried out.

The prevent invention is not restricted to the aforementioned description having made with reference to embodiments. It contains a great number of variations without departing from the technological concept or spirit of the prevent invention. For example, it goes without saying that the medical image generating apparatus 1 can be a radiation image radiographic apparatus using an X-ray flat panel disk, a computer tomography (CT), magnetic resonance imaging (MRI), digital radiographic apparatus (DR) and ultra sound (US) diagnostic apparatus, in addition to the CR apparatus designed to read out the radiation image from the stimulable phosphor panel where the information on patient's radiation image is recorded. Further, these apparatuses can be connected to the medical network system of the present invention.

Effects of the Invention

The medical image processing apparatus, medical network system and program for medical image processing apparatus of the present invention allow the order of the images to be corrected to be suitable for observation of images on the image display device, thereby ensuring proper diagnosis in a manner that facilitates doctor's diagnosis.

What is claimed is:

1. A medical image processing apparatus for receiving medical image information from another apparatus, performing an image processing and sending a medical image to another device to display, comprising:
a first specifying tool for specifying a first display order of the medical image;
a second specifying tool for specifying information to be used for determining a display order of the medical image on a display terminal; and
a correcting tool for correcting the information to be used for determining the display order to conform to the first display order.

2. The medical image processing apparatus of claim 1, wherein the first specifying tool comprises:
a display for displaying a received medical image in a thumbnail form or in a list, for each examination; and
a specifying tool for sorting the displayed thumbnail or the displayed list and specifying the first display order of the medical image.

3. The medical image processing apparatus of claim 1, wherein the first specifying tool is a tool for indicating a permission of starting image output for each image in an examination, and the correcting tool corrects the information to be used for determining the display order to conform to an order in which the permission of starting image output is given in the examination.

4. The medical image processing apparatus of claim 1, wherein the first specifying tool is a tool for receiving request information and determining an order of the medical image requested in an examination, according to the request information received; wherein the correcting tool corrects the information to be used for determining the display order to conform to the order determined according to the request information.

5. The medical image processing apparatus of claim 1, wherein the first specifying tool comprises:
a storing tool for storing a plurality of condition files, in each of the plurality of condition files information for image processing and outputting of an image file being previously set;
a presetting tool for presetting an order of images in each of the plurality of condition files;
a determining tool for determining a condition file to be used for a received medical image, from among the plurality of condition files;
the correcting tool corrects the information to be used for determining the display order to conform to an order specified by the condition file determined.

6. The medical image processing apparatus of claim 1, wherein information on series is changed after the display order at the display terminal has been sorted according to information on a site to be diagnosed.

7. A medical network system comprising:
a medical image generating apparatus for generating medical image information;
a medical image processing apparatus for receiving the medical image information from the medical image generating apparatus, performing an image processing; and
a display terminal for displaying the medical image sent from the medical image processing apparatus,
wherein the medical image processing apparatus comprising:
a first specifying tool for specifying a first display order of the medical image;
a second specifying tool for specifying information to be used for determining a second display order of the medical image on the display terminal; and
a correcting tool for correcting the information to be used for determining the second display order to conform to the first display order.
